(19) United States
(12) Patent Application Publication
(10) Pub. No.: US 2016/0317232 A1
(43) Pub. Date: Nov. 3, 2016

(54) MEDICAL IMAGING PROBE INCLUDING AN IMAGING SENSOR

(71) Applicant: GENERAL ELECTRIC COMPANY, Schenectady, NY (US)

(72) Inventors: Régis VAILLANT, Buc (FR); Olivier GERRARD, Buc (FR)

(21) Appl. No.: 15/109,083

(22) PCT Filed: Dec. 30, 2013
(86) PCT No.: PCT/IB2013/003059

§ 371 Date: Jun. 29, 2016

(51) Int. Cl. A61B 34/20; A61B 6/12; A61B 6/00; A61B 8/00

(52) U.S. Cl. CPC A61B 34/20; A61B 8/4444; A61B 8/4254; A61B 6/12; A61B 8/4263; A61B 6/5247; A61B 2034/2046

(57) ABSTRACT

This disclosure relates to a medical imaging probe comprising: a head enclosed by an inner casing and including an imaging sensor, a cable connected to the head, an outer casing attached to and enclosing the inner casing, and wherein the outer casing is physically connected to at least one localization sensor.

### FIELD OF THE INVENTION

The invention relates to medical imaging probes which include an imaging sensor. The invention more particularly also relates to medical imaging probes which include an ultrasound imaging sensor associated to an X-ray imaging device external to the medical imaging probe.

### BACKGROUND OF THE INVENTION

A problem which may rise when using a medical imaging probe is to be able to place correctly the images shot by the medical imaging probe in a given referential. This may become especially critical, when the move of the medical imaging probe is quite complex, what is for example the case of a medical imaging probe moving within a patient body. This given referential may be a referential which will be common to successive images of a same region of a patient body or may be a referential which will be common with one or more images coming from another imaging device which either is stationary or moves completely independently of the medical imaging probe. To solve this problem, localization of the medical imaging probe needs to be placed in a wished referential which is either absolute or relative to images coming from another imaging device external to the medical imaging probe.

According to a first prior art, for example described in patent application US 2012/0245458, it is known taking a fluoroscopic image of the medical imaging probe, and by analyzing this fluoroscopic image, deducing the localization of the medical imaging probe. A first drawback of this first prior art is a limited precision got from the calculated localization of the medical imaging probe. A second drawback of this first prior art comes from the need of an external device to perform the fluoroscopic image.

According to a second prior art, for example described in patent U.S. Pat. No. 7,713,210, it is known integrating localization sensors within the inner casing of an ultrasound catheter. Integrating such localization sensors within the inner casing of a medical imaging probe, especially if as sophisticated as a transesophageal echocardiographic probe, is quite difficult and will require complete redesign of existing medical imaging probes as well as numerous complex validation steps to be performed again. Besides, all existing medical imaging probes would need to be replaced in order to benefit from this new functionality. All the more that this localization of the medical imaging probe may not always be needed, i.e. not needed each time it is used.

### SUMMARY OF THE INVENTION

The object of the present invention is to alleviate at least partly the above mentioned drawbacks.

More particularly, embodiments of the invention aims to provide a medical imaging probe, including localization capabilities, without needing to redesign completely the existing medical imaging probes, these localization capabilities being implemented in a simple and easy way. This technical problem has absolutely not been tackled by the known prior art.

Therefore, the medical imaging probe provided by embodiments of the invention proposes surrounding and tightening the head of an existing medical imaging probe, already integrated in an inner casing, by an outer casing integrating localization sensor(s).

Embodiments of this invention are solving the problem of repositioning the ultrasound images coming from the medical imaging probe's head in a fixed referential which is independent of the medical imaging probe position and orientation.

Making this outer casing removable from the rest of the head of the medical imaging probe is proposed by embodiments of the invention in order to minimize interferences with the sophisticated and optimized structure of an existing medical imaging probe, what is advantageous because it is simpler to implement.

This object is achieved with a medical imaging probe comprising a head enclosed by an inner casing and including an imaging sensor, a cable connected to said head, an outer casing attached to and enclosing said inner casing, and wherein said outer casing is physically connected to at least one localization sensor.

This object is also achieved with a medical imaging system comprising a medical imaging probe according to any of preceding claims, and a microprocessor coupled to said probe so as to dispose, in a common referential, several images transmitted by said probe. Said medical imaging system, not associated with an X-ray imaging device, is preferably a surgical system or a diagnostic system. When it is used in setting without an X-ray interventional imaging system, the position and orientation of the ultrasound images are known with respect to the localization sensors. So, since position and orientation of the localization sensors with respect to the table supporting the patient are got from the localization sensors themselves, the ultrasound images can be presented with information indicating their position and orientation relative to the patient.

This object is also achieved with a medical imaging system comprising a medical imaging probe according to any of preceding claims, an X-ray imaging device, a microprocessor coupled to said probe and to said X-ray imaging device so as to dispose, in a common referential, at least an image transmitted by said probe and an image transmitted by said X-ray imaging system, both images having been shot at the same time. Preferably, both images shot at the same time correspond to a common region of a body of a patient. Preferably, several couples of both images shot at the same time are disposed in said common referential. Said medical imaging system, associated to an X-ray imaging device is preferably an interventional system.

In the field of cardiac interventional procedures, proposed invention is especially interesting. Indeed, there are a growing number of cardiac procedures which are guided through the combined use of transesophageal ultra-sound imaging and X-ray guidance. By now, each of these two systems is able to deliver real time images of the patient anatomy. These images are complementary because they offer different views of this anatomy. A potential weakness of this combined imaging system is that the obtained pieces of information are not spatially related. The X-ray images are realized in relation with the geometry of the X-ray imaging chain comprising a gantry holding on one side an X-ray tube and on the other side a detector. The transesophageal ultra-sound images are delivered in relationship with the position and orientation of the probe. The probe is introduced in the esophagus of the patient. So, there is no direct and immediate way to relate these two datasets from a pure geometrical point of view.

Ultra-sound and X-ray images complementary aspect, is hereby explained. In cardiology, the physician often has access to real time X-ray images acquired by a C-arm. These images have a good spatial and temporal accuracy thereby enabling to follow precisely the progression of thin catheters and other interventional tools like a medical imaging probe. However, soft human tissues are barely visible in these images, and furthermore, these images are projections which do not give a direct access to the volumetric geometry of the intervention scene. To gain access to this interesting information, a solution consists in using a second imaging modality which is both three dimensional and able to image soft human tissues. A possible choice for this second imaging system is three dimensional ultrasound imaging. This modality allows for been used in real time during the surgical procedure. In cardiologic procedure, transesophageal probes can be navigated right next to the heart, producing real time volumetric images with anatomical details that would be hardly visible with transthoracic ultrasound probes.

This object is also achieved with an outer casing of a medical imaging probe integrating one or more localization sensors and being shaped so as to surround and tighten an inner casing of a head of said medical imaging probe including an imaging sensor.

This object is also achieved with a preparation method of a medical imaging probe which includes a head integrated in an inner casing and including an imaging sensor and which includes a cable connected to said head, comprising a step of surrounding and tightening said inner casing by an outer casing integrating one or more localization sensors.

Preferred embodiments comprise one or more of the following features, which can be taken separately or together, either in partial combination or in full combination; these features can also be combined with any of preceding elements achieving the object of the invention.

Preferably, said outer casing is removable. A completely removable outer casing implies reduced interactions with the inner casing of the head: the less one touches to the existing and optimized structure of the head of the medical imaging probe, which is a very sophisticated device, the better. Besides, the medical imaging probe may be used with or without the outer casing at will. Moreover, the head and the outer casing may be cleaned separately, improving thereby the cleaning of the medical imaging probe.

Preferably, said outer casing presents an opening which can be elastically expanded by external action before shrinking back automatically by itself. The mounting of the outer casing is thereby made easier, and especially not even the inner casing of the head of the medical imaging probe needs to be modified. Again, the less one touches to the existing and optimized structure of the head of the medical imaging probe, which is a very sophisticated device, the better.

Preferably, said localization sensor is fully encompassed in a wall of said outer casing. This a simple and practical way to implement the localization sensors which on the one hand do not interfere in the least with the inner casing of the head and on the other hand will not interfere at all with the body of the patient in which they may be introduced during performance of imaging.

Preferably, it comprises several said localization sensors, preferably at least three or four localization sensors, disposed at different locations of said outer casing. Therefore, localization of the medical imaging probe will be more accurate, improving its disposition in the wished referential.

Preferably, said localization sensor is adapted to transmit wirelessly its localization data. Thereby, no modification, and especially no diameter widening of the cable of the medical imaging probe is needed.

Preferably, said localization sensor is power alimented by a battery integrated in said outer casing and independent of said head. This allows for powering the localization sensor(s) again without interfering with the head or the cable of the medical imaging probe. An alternative would be implementing a derivation of the alimentation wire of the head which is located in the cable, near the head, so as to redirect part of the incoming power for powering the localization sensor(s). More preferably, said battery is wirelessly rechargeable. Source of energy recharging said battery may be located either in the medical imaging probe or outside the medical imaging probe.

Preferably said localization sensor is wirelessly power alimented from outside said outer casing. Source of energy of such wireless power alimentation can be located either in the medical imaging probe or outside the medical imaging probe.

Preferably, said localization sensor is adapted to be wirelessly activated and deactivated. This allows for easily saving energy of the battery located in the outer casing. This is all the more interesting that this battery is an independent and autonomous battery located in the outer casing.

Preferably, said localization sensor is a magnetic sensor. The magnetic sensor presents the advantage of providing very accurate localization data, especially when several such magnetic sensors are used. So, preferably, said localization sensor is a magnetic sensor; alternatively, said localization sensor may be an electric sensor or an ultrasound sensor. These magnetic sensors are associated to several magnetic emitters, preferably three of them, disposed for example under the examination table. Localization data of each magnetic sensor may then be calculated by triangulation.

Preferably, said probe is a transesophageal echocardiographic probe. Instead of a transesophageal echocardiographic probe, a transesophageal echographic probe or a transesophageal ultrasound probe can be used. Due to very sophisticated structure of existing transesophageal echocardiographic probe, because of the high level of constraints, providing a separable outer casing is very interesting, since trying to integrate the localization sensors directly within the inner casing of the head of the medical imaging probe would imply redesigning completely this medical imaging probe, what would be difficult and expensive. Said probe may be alternatively a transthoracic echocardiographic probe. However, the benefit brought by invention implementation is not as important as in the case of a transesophageal echocardiographic probe, because being not expected to be introduced within the body of the patient, this transthoracic echocardiographic probe has to meet fewer constraints than a transesophageal echocardiographic probe.

Preferably, said head is larger than said cable, said head width ranging preferably from 5 to 10 mm. Head being larger than cable helps to dispose the outer casing around the inner casing in such a way that the outer casing does not move relatively to the inner casing afterwards or does not fall off the inner casing, without needing any modification of the inner casing to attach parts of the outer casing on the inner casing.

Preferably, said outer casing material is a plastic.

Preferably, said outer casing surrounds and tightens part of said cable too. This again helps to dispose the outer casing around the inner casing in such a way that the outer casing does not move relatively to the inner casing afterwards or does not fall off the inner casing, without needing any modification of the inner casing to attach parts of the outer casing on the inner casing.

Preferably, either said outer casing surrounds and tightens all the inner casing of said head except for a sensitive surface of said imaging sensor, or said outer casing surrounds and tightens all the inner casing of said head, part of said outer casing surrounding a sensitive surface of said imaging sensor being transparent to signal emitted and/or received by said imaging sensor. Thereby, the outer casing may be well attached around the inner casing, while offering several different locations to dispose respectively different localization sensors, and without disturbing the signal emitted by or received on the sensitive surface of the imaging sensor.

Preferably, said outer casing has a thickness ranging from 2 mm to 4 mm. This thickness ranges is sufficient to integrate the localization sensors, but at the same time kept relatively low in order not to impede the move of the medical imaging probe, especially within the body of the patient.

Preferably, wirelessly is short range wirelessly, for example less than 10 m, more preferably less than 5 m, even more preferably less than 1 m. In case the control system receiving the localization data from the localization sensor(s) is quite close to the patient, which may especially be the case with magnetic sensor(s), only a very limited range for wireless communication is required.

Preferably, localization data transmitted by said localization sensor include position and/or orientation of said localization sensor. This allows for a more precise and more complete localization of the medical imaging probe with respect to the wished referential.

A casing is defined for the medical imaging probe, whose objective is allowing for disposing the ultrasound images. This casing presents preferably several localization sensors. The localization sensors will indicate the position and orientation of the casing and hence the position and orientation of the medical imaging probe. By merging this information with the geometry of the ultrasound beam relative to the medical imaging probe, the ultrasound images can then be related to the referential defined by the localization sensors.

Preferably, said imaging sensor is an ultrasound sensor. This allows for complete imaging data, especially when coupled with an X-ray imaging device.

Further features and advantages of the invention will appear from the following description of embodiments of the invention, given as non-limiting examples, with reference to the accompanying drawings listed hereunder.

### BRIEF DESCRIPTION OF THE DRAWINGS

FIG. 1 shows schematically an example of a medical imaging probe according to embodiments of the invention.

### DETAILED DESCRIPTION OF THE INVENTION

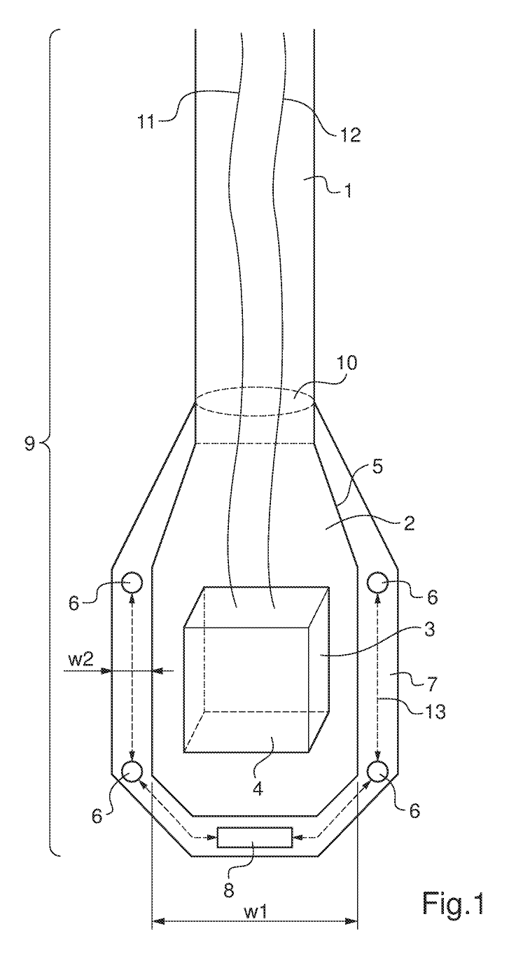

FIG. 1 shows schematically an example of a medical imaging probe according to embodiments of the invention. The medical imaging probe 9 comprises a cable 1 at one end of which is fixed a head 2. Cable 1 comprises a data wire 11 and a power wire 12. The medical imaging probe 9 is preferably a transesophageal echocardiographic probe.

The head 2 is surrounded by an inner casing 5. The head 2 comprises an imaging sensor 3. Preferably, imaging sensor 3 is an ultrasound sensor. The imaging sensor 3 comprises a sensitive surface 4. Imaging signal emission and reception between the imaging sensor 3 and the object to be imaged are performed through this sensitive surface 4. The head 2 presents a width w1. Head 2 is larger than cable 1. Head 2 width w1 ranges preferably from 5 to 10 mm. Data wire 11 and a power wire 12 are both connected to imaging sensor 3 within head 2.

The inner casing 5 is itself surrounded by an outer casing 7. This outer casing 7 is preferably made of an elastic material so as to be slipped on the inner casing 5. Therefore, an opening 10 in outer casing 7 can be temporarily widened by stretching. This elasticity allows for outer casing 7 to surround and tighten inner casing 5 after inner casing 5 has been introduced within outer casing 7 through opening 10. Outer casing 7 surrounds and tightens part of cable 1 too. Outer casing 7 presents a thickness w2. Preferably, outer casing 7 has a thickness w2 ranging from 2 mm to 4 mm, for example about 3 mm. Outer casing 7 is removable from head 2.

Several localization sensors 6, preferably 3 or 4 of them, are fully embedded within the wall of outer casing 7. Those localization sensors 6 are disposed at different locations of said outer casing 7. Those localization sensors 6 are well distributed in the outer casing 7. Those localization sensors 6 will transmit permanently or almost permanently their position and/or orientation so that the position and orientation of the probe 9 can be deduced therefrom. Those localization sensors 6 transmit their localization data wirelessly. Besides, the localization sensors 6 are adapted to be wirelessly activated and deactivated. Those localization sensors 6 are linked via electrical wires 13 to a battery 8 which is also embedded within the wall of outer casing 7. The localization sensors 6 are power alimented by this battery 8 which is integrated in outer casing 7 and which is independent of head 2. Preferably, the localization sensors 6 are magnetic sensors. The localization sensors 6 could also be electric and ultrasound sensors as an alternative.

Outer casing 7 allows for the signal emitted by imaging sensor 3 being transmitted through sensitive surface 4. Therefore, either part of outer casing 7 facing sensitive surface 4 is transparent to radiation emitted by imaging sensor 3, or there is a hole in outer casing 7 facing sensitive surface 4 which second alternative is less satisfactory with respect to mechanical progression of probe 9 within the human body.

The invention has been described with reference to some preferred embodiments. However, many variations are possible within the scope of the invention.

1. A medical imaging probe comprising:
a head enclosed by an inner casing and including an imaging sensor;
a cable connected to the head; and
an outer casing attached to and enclosing the inner casing, wherein the outer casing is physically connected to at least one localization sensor.

2. The medical imaging probe according to claim 1, wherein said the outer casing is removable.

3. The medical imaging probe according to claim 1, wherein the outer casing presents an opening which can be elastically expanded by external action before shrinking back automatically by itself.

4. The medical imaging probe according to claim 1, wherein the at least one localization sensor is fully encompassed in a wall of the outer casing.

5. The medical imaging probe according to claim 1 comprising a plurality of localization sensors, disposed at different locations about the outer casing.

6. The medical imaging probe according to claim 1, wherein the at least one localization sensor is configured to transmit wirelessly its localization data.

7. The medical imaging probe according to claim 1, wherein the at least one localization sensor is power alimented by a battery integrated in said the outer casing and independent of the head.

8. The medical imaging probe according to claim 1, wherein the at least one localization sensor is wirelessly power alimented from outside the outer casing.

9. The medical imaging probe according to claim 1, wherein the at least one localization sensor is configured to be wirelessly activated and deactivated.

10. The medical imaging probe according to claim 1, wherein the at least one localization sensor is a magnetic sensor.

11. The medical imaging probe according to claim 1, wherein the probe is an ultrasound probe or an echographic probe.

12. The medical imaging probe according to claim 1, wherein the head is larger than the cable, the head width ranging preferably from about 5 mm to about 10 mm.

13. A medical imaging system comprising:
a medical imaging probe comprising:
a head enclosed by an inner casing and including an imaging sensor;
a cable connected to the head; and
an outer casing attached to and enclosing the inner casing,
wherein the outer casing is physically connected to at least one localization sensor; and
a microprocessor coupled to the probe so as to dispose, in a common referential, several images transmitted by the probe.

14. The medical imaging system of claim 13, further comprising:
an X-ray imaging device,
wherein the microprocessor is further coupled to the X-ray imaging device so as to dispose, in a common referential, at least an image transmitted by the probe and an image transmitted by the X-ray imaging system, both images having been shot at the same time.

15. Outer casing of a medical imaging probe The medical imaging probe according to claim 1, wherein the at least one localization sensor is integrated into the outer casing, the outer casing being shaped so as to surround and tighten the inner casing of the head of the medical imaging probe including the imaging sensor.

16. (canceled)

17. The medical imaging probe according to claim 7, wherein the battery is wirelessly rechargeable.
